(19) United States
(12) Patent Application Publication
(10) Pub. No.: US 2024/0105312 A1
(43) Pub. Date: Mar. 28, 2024

(54) COMPUTED TOMOGRAPHY IMAGING METHOD AND APPARATUS

(71) Applicant: GE Precision Healthcare LLC, Waukesha, WI (US)

(72) Inventors: Ximiao Cao, Beijing (CN); Jingting Li, Beijing (CN); Bingjie Zhao, Beijing (CN)

(21) Appl. No.: 18/473,983

(22) Filed: Sep. 25, 2023

(30) Foreign Application Priority Data
Sep. 26, 2022 (CN) 202211171872.5

(51) Int. Cl. G16H 30/20; G06T 7/00

(52) U.S. Cl. CPC G16H 30/20; G06T 7/00; G06T 2207/10081

(57) ABSTRACT

Embodiments of the present invention provide a computed tomography (CT) imaging method and apparatus. The CT imaging method includes performing a three-dimensional scan of an examined site to obtain a three-dimensional scan image of the examined site and attenuation information of the examined site, the three-dimensional scan image being used to position a scan range. The method further includes calculating the tube current profile of an X-ray radiation dose for the examined site according to the three-dimensional scan image and attenuation information, and performing a main scan of the examined site according to the tube current profile to obtain a main scan image.

### CROSS-REFERENCE TO RELATED APPLICATIONS

This application claims priority to Chinese Application No. 202211171872.5, filed on Sep. 26, 2022, the disclosure of which is incorporated herein by reference in its entirety.

### TECHNICAL FIELD

Embodiments of the present invention relate to the technical field of medical devices, and in particular to a computed tomography imaging method and apparatus.

### BACKGROUND

CT scanning, i.e., computed tomography, utilizes a precisely collimated X-ray beam or the like, and a highly sensitive detector to scan cross sections of a certain site of a human body one by one, having the features of short scanning time, image clarity, etc., and can be used to detect a wide range of diseases.

Traditional CT scan processes include two scans, the first scan being a two-dimensional scout scan for positioning a scan range, and the second scan being a main scan for imaging. The two-dimensional scout scan is a radiogram scan of a patient before the main scan, and the obtained positioning image provides the basis for patient scan range selection and automatic exposure control (AEC). In accordance with a pre-calculated tube current (mA) profile of an X-ray tube, the tube current can be adapted to a patient within the scan range during scanning, the mA profile being a series of mA values that vary with scan positions and rotation angles.

It should be noted that the above description of the background is only for the convenience of clearly and completely describing the technical solutions of the present application and to facilitate the understanding of those skilled in the art.

### SUMMARY

The inventors found that, conventionally, a pre-prepared scan (e.g., a two-dimensional scout scan) at a fixed angle (e.g., a front-rear/rear-front, and/or transverse) is used to position a scan range and a critical organ, and provide patient position and attenuation information for a mA profile estimation in a scanning plan of a main scan. However, although the dose for the two-dimensional scout scan is only a small portion of the dose of the main scan, improper centering thereof causes the problem of patient size ambiguity in an image, and tissue overlap in an image causes the problem of difficulty in organ segmentation.

With respect to at least one of the above technical problems, embodiments of the present application provide a computed tomography imaging method and apparatus so as to solve the problems of patient size ambiguity in an image and difficulty in organ segmentation by providing information of other projection angles, by means of replacing a two-dimensional scout scan with a three-dimensional scan.

According to one aspect of the embodiments of the present application, a computed tomography imaging method is provided. The method includes performing a three-dimensional scan of an examined site to obtain a three-dimensional scan image of the examined site and attenuation information of the examined site, the three-dimensional scan image being used to position a scan range, calculating a tube current profile of an X-ray radiation dose of the examined site according to the three-dimensional scan image and attenuation information, and performing a main scan of the examined site according to the tube current profile to obtain a main scan image.

In some embodiments, the X-ray radiation dose used for the three-dimensional scan is within 4% of the X-ray radiation dose used for the main scan. In some embodiments, the difference between the X-ray radiation dose used for the three-dimensional scan and 1% of the X-ray radiation dose used for the main scan is within a predetermined range. In some embodiments, the pitch factor of a pitch used for the three-dimensional scan is greater than 1.0. In some embodiments, the method further includes extracting the attenuation information for all angles in the scan range from raw scan data and the three-dimensional scan image obtained by carrying out the three-dimensional scan of the examined site. In some embodiments, calculating the tube current profile includes performing organ segmentation on the three-dimensional scan image to generate a three-dimensional organ picture, and calculating the tube current profile along the scan range according to the three-dimensional organ picture and attenuation information corresponding to the three-dimensional organ picture.

In some embodiments, the three-dimensional scan is a spiral scan. According to another aspect of the embodiments of the present application, a computed tomography imaging apparatus is provided, which includes a first scan unit that performs a three-dimensional scan of an examined site, to obtain a three-dimensional scan image of the examined site and attenuation information of the examined site, the three-dimensional scan image being used for positioning a scan range, an automatic exposure control (AEC) unit that calculates the tube current profile of an X-ray radiation dose of the examined site according to a three-dimensional scout scan image and the attenuation information, and a second scan unit that performs a main scan of the examined site according to the tube current profile to obtain a main scan image.

In some embodiments, the X-ray radiation dose used for the three-dimensional scan is within 4% of the X-ray radiation dose used for the main scan. In some embodiments, the difference between the X-ray radiation dose used for the three-dimensional scan and 1% of the X-ray radiation dose used for the main scan is within a predetermined range. In some embodiments, the pitch factor of a pitch used for the three-dimensional scan is greater than 1.0.

In some embodiments, the apparatus further includes an extraction unit that extracts the attenuation information for all angles in the scan range from raw scan data and the three-dimensional scout scan image obtained by carrying out the three-dimensional scan of the examined site. In some embodiments, the device further includes a segmentation unit that performs organ segmentation on the three-dimensional scan image to generate a three-dimensional organ picture. In the above embodiment, the AEC unit calculating the tube current profile along the scan range according to the three-dimensional organ picture and the attenuation information corresponding to the three-dimensional organ picture. In some embodiments, the three-dimensional scan is a spiral scan.

According to another aspect of the embodiments of the present application, an electronic device is provided which includes a memory and a processor, the memory storing a computer program, and the processor being configured to execute the computer program so as to implement the described computed tomography imaging method.

One of the benefits of the embodiments of the present application is, according to the embodiments of the present application, the problem of patient size ambiguity in an image caused by improper centering during a two-dimensional scout scan is prevented by replacing a two-dimensional scout scan with a three dimensional scan, thereby providing precise three-dimensional organ segmentation for organ-based mA (X-ray tube current) modulation and organ protection, thus avoiding the problem of difficulty in organ segmentation in the image.

With reference to the following description and drawings, specific implementations of the embodiments of the present application are disclosed in detail, and the means by which the principles of the embodiments of the present application can be employed are illustrated. It should be understood that the embodiments of the present application are not limited in scope thereby. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of embodiments of the present application, which constitute a part of the description and are used to illustrate embodiments of the present application and explain the principles of the present application together with textual description. Evidently, the drawings in the following description are merely some embodiments of the present application, and a person of ordinary skill in the art may obtain other embodiments according to the drawings without involving inventive skill. In the drawings:

FIG. 1 is a schematic diagram of a CT imaging device according to an embodiment of the present application;

FIG. 2 is a schematic diagram of a CT imaging system according to an embodiment of the present application;

FIG. 3 is a schematic diagram of a computed tomography imaging method according to an embodiment of the present application;

FIG. 4 is a schematic diagram of an example of a noise model;

FIG. 5 is another schematic diagram of a computed tomography imaging method according to an embodiment of the present application;

FIG. 6 is a schematic diagram of a computed tomography imaging apparatus according to an embodiment of the present application; and

FIG. 7 is a schematic diagram of an electronic device according to an embodiment of the present application.

### DETAILED DESCRIPTION

The foregoing and other features of the embodiments of the present application will become apparent from the following description and with reference to the drawings. In the description and drawings, specific embodiments of the present application are disclosed in detail, and part of the implementations in which the principles of the embodiments of the present application may be employed therein are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise,” “include,” “have” etc., refer to the presence of described features, elements, components, or assemblies, but do not exclude the presence or addition of one or more other features, elements, components, or assemblies.

In the embodiments of the present application, the singular forms “a” and “the”, etc. include plural forms, and should be broadly construed as “a type of” or “a class of” rather than being limited to the meaning of “one.” Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ,” unless otherwise specified in the context.

The features described and/or illustrated for one embodiment may be used in one or more other embodiments in the same or similar manner, be combined with features in other embodiments, or replace features in other embodiments. The term “include/comprise” when used herein refers to the presence of features, integrated components, steps, or assemblies, but does not preclude the presence or addition of one or more other features, integrated components, steps, or assemblies.

The device described herein that obtains medical imaging data may be applicable to various medical imaging modalities, including but not limited to, CT (computed tomography) devices, PET (positron emission tomography)-CT, or any other suitable medical imaging devices.

The system obtaining the medical imaging data may include the aforementioned medical imaging device, and may include a separate computer device connected to the medical imaging apparatus, and may further include a computer device connected to an Internet cloud, the computer device being connected by means of the Internet to the medical imaging apparatus or a memory for storing medical images. The imaging method may be independently or jointly implemented by the aforementioned medical imaging device, the computer device connected to the medical imaging device, and the computer device connected to the Internet cloud.

As an example, the embodiments of the present application are described below in conjunction with an X-ray computed tomography (CT) device. Those skilled in the art will appreciate that the embodiments of the present application can also be applied to other medical imaging devices.

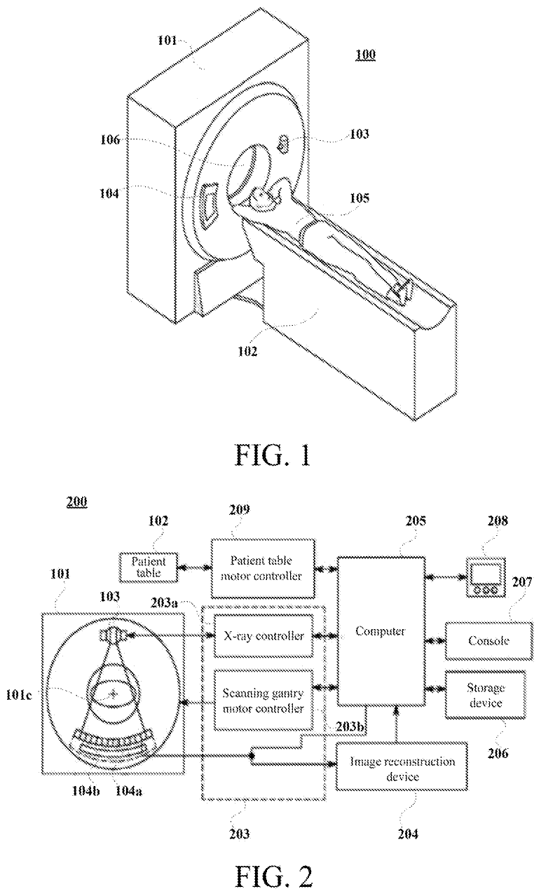

FIG. 1 is a schematic diagram of a CT imaging device according to an embodiment of the present application, schematically showing the situation of a CT imaging device 100. As shown in FIG. 1, the CT imaging device 100 includes a scanning gantry 101 and a patient table 102; the scanning gantry 101 has an X-ray source 103 projecting an X-ray beam towards a detector assembly or collimator 104 on an opposite side of the scanning gantry 101. A test object 105 can lie flat on the patient table 102 and be moved into a scanning gantry opening 106 along with the patient table 102. Medical imaging data of the test object 105 can be acquired by means of a scan carried out by the X-ray source 103.

FIG. 2 is a schematic diagram of a CT imaging system according to an embodiment of the present application, schematically showing a block diagram of a CT imaging system 200. As shown in FIG. 2, the detector assembly 104 includes a plurality of detector units 104a and a data acquisition system (DAS) 104b. The plurality of detector units 104a sense a projected X-ray passing through the test object 105.

The DAS 104b, according to the sensing of the detector units 104a, converts collected information into projection data for subsequent processing. During the scanning for acquiring the X-ray projection data, the scanning gantry 101 and components mounted thereon rotate around a rotation center 101c.

The rotation of the scanning gantry 101 and the operation of the X-ray source 103 are controlled by a control mechanism 203 of the CT imaging system 200. The control mechanism 203 includes an X-ray controller 203a that provides power and a timing signal to the X-ray source 103 and a scanning gantry motor controller 203b that controls the rotational speed and position of the scanning gantry 101. An image reconstruction apparatus 204 receives the projection data from the DAS 104b and performs image reconstruction. A reconstructed image is transmitted as an input to a computer 205, and the computer 205 stores the image in a mass storage apparatus 206.

The computer 205 also receives commands and scanning parameters from an operator by means of a console 207. The console 207 has an operator interface of a certain form, such as a keyboard, a mouse, a voice activated controller, or any other suitable input device. An associated display 208 allows the operator to observe the reconstructed image and other data from the computer 205. The commands and parameters provided by the operator are used by the computer 205 to provide control signals and information to the DAS 104b, the X-ray controller 203a, and the scanning gantry motor controller 203b. Additionally, the computer 205 operates a patient table motor controller 209 which controls the patient table 102 so as to position the test object 105 and the scanning gantry 101. In particular, the patient table 102 moves the test object 105 as a whole or in part to pass through the scanning gantry opening 106 in FIG. 1.

The foregoing schematically illustrates the device and the system for acquiring medical imaging data (or also referred to as medical images or medical image data) according to the embodiments of the present application, but the present application is not limited thereto. The medical imaging device may be a CT device, a PET-CT, or any other suitable imaging device. A storage device may be located within the medical imaging device, in a server outside the medical imaging device, in an independent medical imaging storage system (such as a Picture Archiving and Communication System (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. A medical image cloud platform analysis system may be positioned away from the medical imaging device, for example, arranged at a cloud end that is in communication with the medical imaging device.

As an example, after a medical institution completes an imaging scan using a medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SAAS).” The SAAS may exist between hospitals, between a hospital and an imaging center, or between a hospital and a third-party online diagnosis and treatment service provider.

The medical image scanning is schematically illustrated above, and the embodiments of the present application are described in detail below in view of the accompanying drawings.

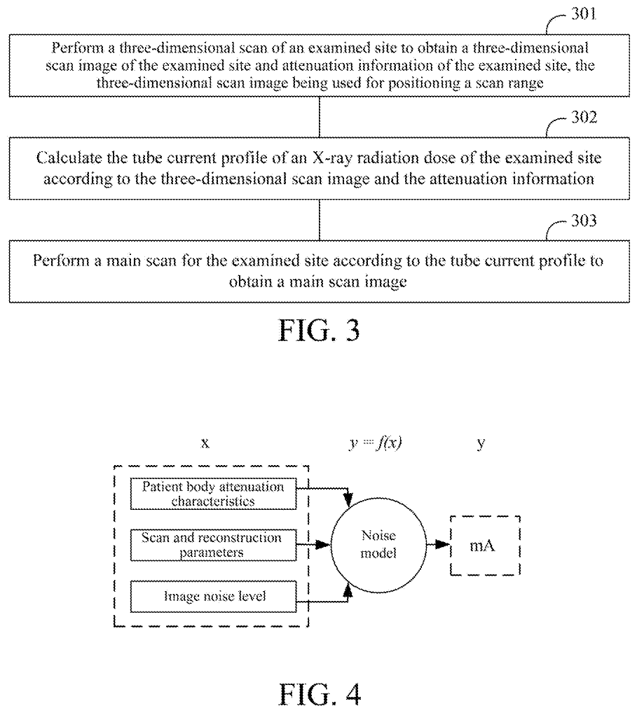

Embodiments of the present application provide a computed tomography imaging method. FIG. 3 is a schematic diagram of a computed tomography imaging method according to an embodiment of the present application, and as shown in FIG. 3, the method includes performing a three-dimensional scan of an examined site to obtain a three-dimensional scan image of the examined site and attenuation information of the examined site, the three-dimensional scan image being used for positioning a scan range (block 301), calculating the tube current profile of an X-ray radiation dose of the examined site according to the three-dimensional scan image and attenuation information (block 302), and performing a main scan of the examined site according to the tube current profile, to obtain a main scan image (block 303).

It should be noted that FIG. 3 merely schematically illustrates the embodiment of the present application, but the present application is not limited thereto. For example, some other operations may also be added, or some of the operations may be omitted. Those skilled in the art can make appropriate variations according to the above content, rather than being limited by the disclosure of FIG. 3.

According to the above embodiments, the problem of patient size ambiguity in an image caused by improper centering during a two-dimensional scout scan is prevented by means of replacing two-dimensional scout scans with three dimensional scans, thereby providing precise three-dimensional organ segmentation for organ-based mA (X-ray tube current) modulation and organ protection, thus avoiding the problem of difficulty in organ segmentation in the image.

In 301, the examined site may be any site of a subject (e.g., a patient), such as the head, chest, waist, leg, and the like. Raw scan data is obtained by performing a three-dimensional scan of the examined site, and the three-dimensional scan image and the attenuation information of the examined site can be obtained on the basis of the raw scan data. The attenuation information refers to information obtained after the X-ray is attenuated by a subject, and its specific definition and acquisition means may refer to related technology, and is merely illustrative herein.

Here, the three-dimensional scan image may be obtained by performing image reconstruction on raw scan data, and the present application does not limit a specific image reconstruction method, and reference may be made to related technology. Moreover, the attenuation information mainly includes the X-ray attenuation amount for scanned tissue having a particular size, shape, position, and material, and can be obtained from the raw scan data, and the three-dimensional scan image.

For example, a total attenuation amount of a ray that penetrates a certain site along a certain direction or at a certain angle is obtained from the raw scan data; and a path length of a ray that penetrates a certain site along a certain direction or at a certain angle and the attenuation coefficient data and profile of the penetrated tissue, are obtained from the three-dimensional scan image, thereby obtaining attenuation information for all angles within the scan range.

In the above embodiments, the scan range may be pre-set or prescribed by an operator or a protocol before scanning, and may be adjusted by an operator after obtaining a three-dimensional scan image, but the present application is not limited thereto.

In some embodiments, the three-dimensional scan is a spiral scan, but the present application is not limited thereto, and the three-dimensional scan may also be another scanning mode capable of obtaining three-dimensional scan images.

In some embodiments, the X-ray radiation dose used for the three-dimensional scan is within 4% of the X-ray radiation dose used for the main scan. For example, the difference between the X-ray radiation dose used for the three-dimensional scan and 1% of the X-ray radiation dose used for the main scan is within a predetermined range, i.e., the X-ray radiation dose used for the three-dimensional scan is about 1% of the X-ray radiation dose used for the main scan. As such, additional volume information is provided for mA estimation at a low dose increment cost, and there will be no radiation effects on the subject (e.g., a patient) due to excessive X-ray radiation doses.

In the above embodiments, the value of a predetermined range is not limited, and may be 0, i.e., the X-ray radiation dose used for the three-dimensional scan is 1% of the X-ray radiation dose used for the main scan, or may be another value less than 1, i.e., the X-ray radiation dose used for the three-dimensional scan is slightly less than or slightly greater than 1% of the X-ray radiation dose used for the main scan, but the present application is not limited thereto.

In some embodiments, the pitch factor of the pitch used for the three-dimensional scan is greater than 1.0, for example 1.3, and further for example 1.531, but the present application is not limited thereto. Thus, in combination with a low dose of X-rays, by means of the three-dimensional scan of an ultra-low dose and a high pitch, the problem of a size estimation error caused by improper centering during the two-dimensional scout scan is eliminated, and the radiation dose is not increased, thus providing additional volume information for the mA profile estimation.

In some embodiments, the X-ray tube current profile may be obtained by performing organ segmentation on the three-dimensional scan image, and performing calculation using the three-dimensional organ picture and the attenuation information of the three-dimensional organ corresponding thereto.

For example, organ segmentation is performed on a three-dimensional scan image to generate a three-dimensional organ picture, and the X-ray tube current profile is calculated along the scan range according to the three-dimensional organ picture and the attenuation information corresponding to the three-dimensional organ picture, such that a reconstructed image or a certain portion of the reconstructed image has a specified noise level or image quality.

In the above example, the organ segmentation method is not limited; for example, conventional image segmentation, artificial intelligence segmentation, etc. can be used. Even with tissue overlapping, organ segmentation can still be performed more easily due to the organ segmentation being performed on a three-dimensional scan image rather than a two-dimensional scout image.

In the above example, the three-dimensional organ picture of a target organ and the attenuation information corresponding to the three-dimensional organ picture may be inputted to a computing module on the basis of the scanning protocol of an organ of interest, so as to calculate the X-ray tube current profile. Here, the scanning protocol may be pre-configured, or may be manually set or edited by an operator, for example, a critical organ is the liver for a liver-related abdominal scanning protocol, and the operator can add or switch to the spleen, and the like. In the embodiments of the present application, the X-ray tube current profile is calculated on the basis of the three-dimensional organ picture, and an X-ray tube current profile for an organ can be obtained so as to facilitate a main scan.

In the above example, the attenuation information corresponding to the three-dimensional organ picture is obtained from the attenuation information of the aforementioned examined site, for example, from the attenuation information for all angles in the aforementioned scan range, but the present application does not limit the acquisition means; for example, coordinates of each point on the three-dimensional organ picture are determined on the basis of the outline of the three-dimensional organ picture, and the attenuation information corresponding to the three-dimensional organ picture is extracted from the above attenuation information on the basis of coordinates of respective points.

In the above example, the X-ray tube current profile may be calculated along the scan range using the pre-established noise model. The present application is not limited thereto. Other models may also be used to calculate the X-ray tube current profile according to different tube current modulation indexes, such as an equivalent current model, a signal-to-noise ratio model, a value model related to noise levels specified by manufacturers, etc., as long as a reconstructed image or a certain portion of the reconstructed image has a specified noise level or image quality.

In the above example, the noise model is a mapping function among a patient body attenuation characteristic, scan and reconstruction parameters, image noise level (as image quality level), and a corresponding mA value. The model may be empirical data or a theoretical calculation, use curve fitting or machine learning, or any combination of the above methods. The patient body attenuation characteristic corresponds to attenuation information of a particular organ, i.e., the total attenuation, position, size, etc. of the organ, and may be obtained from the three-dimensional scan, for example, from the aforementioned raw scan data and three-dimensional scan image. The scan and reconstruction parameters are pre-set, and may be set manually, or may be automatically set according to information obtained by means of the aforementioned three-dimensional scan. The value of the image noise level may be pre-set or manually set by the operator.

FIG. 4 is a schematic diagram of an example of a noise model. As shown in FIG. 4, the input of the noise model is the patient body attenuation characteristic, the scan and reconstruction parameters, and the image noise level, and the output of the noise model is an mA value. The mA value can be represented by means of an mA profile, and the outputted mA profile includes a series of mA values as a function of scan positions and angles, and is expressed as follows:

mA(z,α)={mA_{z}_{₁}_{,α}_{₁},mA_{z}_{₂}_{,α}_{₂}, . . . , mA_{z}_{ₙ}_{,α}_{ₙ}}

In some examples, in order to derive the noise model, the noise level of a PP mold (circular or oval, etc.) or a body mold (a human body mold) may be measured on the basis of the three-dimensional scan image. A series of measurements are performed for a series of PP body molds having different water equivalent diameters (WED), each measurement using a series of mA values. Multi-dimensional curve fitting is derived from measurements in the following form:

mA=A(WED)·Noise^{B(WED)}

A and B are exponential coefficients and fitted as a WED function.

In order to more clearly understand the method of the embodiments of the present application, the method is described below in view of a specific example.

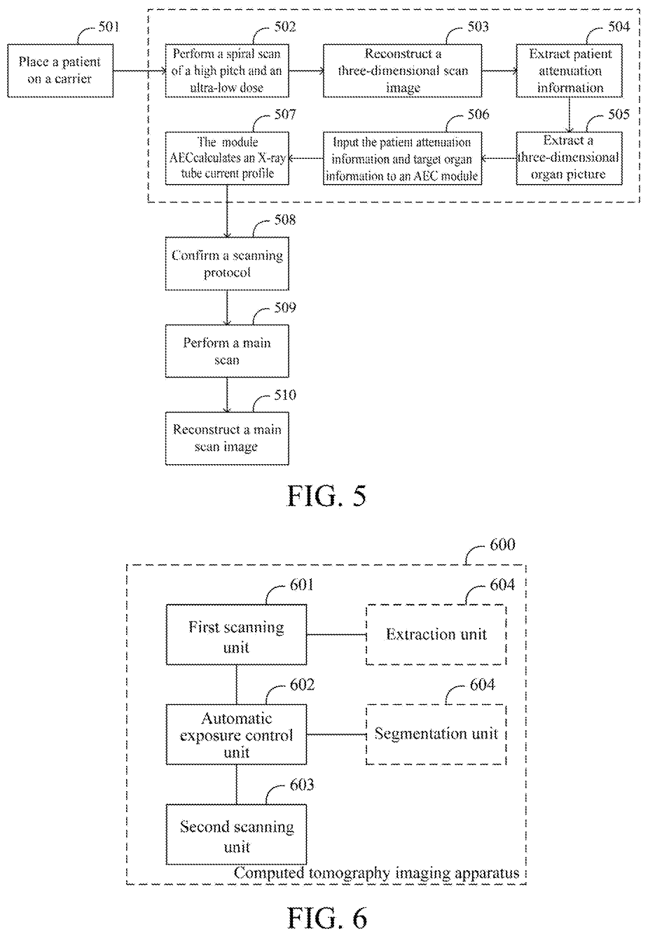

FIG. 5 is another schematic diagram of a computed tomography imaging method according to an embodiment of the present application. As shown in FIG. 5, the method includes placing a patient on a carrier (block 501), performing a spiral scan on the patient using a high pitch and an ultra-low dose (block 502), reconstructing a three-dimensional scan image on the basis of raw scan data (block 503), extracting patient attenuation information from the raw scan data and the three-dimensional scan image (block 504), extracting a three-dimensional organ picture by performing organ segmentation on the three-dimensional scan image (block 505), according to a scanning protocol, inputting the patient attenuation information and target organ information into an AEC (automatic exposure control) module (block 506), the AEC module calculating an X-ray tube current profile (block 507), confirming a scanning protocol corresponding to the target organ (block 508), performing a main scan using the calculated X-ray tube current profile on the basis of the above-described scanning protocol (block 509), and reconstructing a main scan image (block 510).

It should be noted that FIG. 5 merely schematically illustrates an embodiment of the present application, but the present application is not limited thereto. For example, the order of execution between operations may be appropriately adjusted. In addition, some other operations may also be added or some operations may be omitted. Those skilled in the art can make appropriate variations according to the above content, rather than being limited by the disclosure of FIG. 5.

In 504 above, patient attenuation information may be extracted from all angles at each position within the scan range, the attenuation information coming from the raw scan data and the three-dimensional scan image. In conjunction with the raw scan data and three-dimensional scan image, the patient attenuation information may be obtained for all angles at each position within the scan range.

For example, the attenuation information (i.e., the attenuation characteristic) mainly includes X-ray attenuation values for scanned tissue having a particular size, shape, position, and material. The three-dimensional scanning image essentially includes the volume size, position, and attenuation of the scanned anatomical structure. Such information can help to further reduce the total dose and optimize the image quality for the organs.

In 506 above, the target organ information (the position, and size of the target organ, etc.) and the attenuation information (total attenuation, path length, etc.) corresponding to the target organ may be inputted to the AEC calculation module according to a pre-selected scanning protocol.

In 507 above, the AEC calculation module may calculate the desired mA profile along the scan range on the basis of a pre-established noise model (not limited thereto).

In the above example, the AEC calculation module is a computing algorithm for estimating the radiation output at a particular angle and position around a patient, so that the image quality level of the main scan image is consistent with the image quality level desired by the user. The core of the algorithm is a noise model, as previously described, which essentially is a mapping function among the patient body attenuation characteristic, scan and reconstruction parameters, image noise (as image quality level), and corresponding mA values.

In the above embodiments, the three-dimensional scan is a spiral scan, but it does not need to reach diagnostic image quality, unlike the main scan. The three-dimensional scan image is obtained by scanning a patient by means of an ultra-low dose (1% of the dose of the main scan (a conventional scan of the same anatomical structure)) and a high pitch (a pitch factor greater than 1). Here, the raw scan data (a raw signal) is first blurred using a mean filter, and then reconstructed using a standard spiral reconstruction algorithm. The volume of the reconstructed image is processed by means of a graphical-structure-based multi-organ positioning algorithm, to generate a three-dimensional organ picture, that is, labeling voxels of each major organ.

In addition, a noise model is established by performing mapping among the size, shape, position, and attenuation of the three-dimensional volume of the scanned anatomical structure or target organ, scanning and reconstruction parameters of the main scan, the image quantum noise level and the tube current. After a pre-determined three-dimensional scan, the three-dimensional scan image and the attenuation information are obtained using raw scan data, thereby calculating a mA profile, and the main scan is performed by using the mA profile, whereby the scanned image obtained by the main scan has the desired image quality.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to the embodiments of the present application, the problem of patient size ambiguity in an image caused by improper centering during a two-dimensional scout scan is prevented by replacing two-dimensional scout scans with three dimensional scans, thereby providing precise three-dimensional organ segmentation for organ-based mA (X-ray tube current) modulation and organ protection, and thus avoiding the problem of difficulties in organ segmentation in the image. Moreover, a three-dimensional scanning mechanism using an ultra-low dose and a high pitch eliminates the problem of patient size ambiguity in an image, providing additional volume information for mA profile estimation at a smaller dose increase cost.

Embodiments of the present application provide a computed tomography imaging device, and content identical to that of the embodiments of the first aspect is not repeated herein.

FIG. 6 is another schematic diagram of a computed tomography imaging apparatus according to an embodiment of the present application. As shown in FIG. 6, the computed tomography imaging apparatus 600 includes a first scan unit 601 that performs a three-dimensional scan of an examined site to obtain a three-dimensional scan of the examined site image and attenuation information of the examined site, the three-dimensional scan image being used for positioning a scan range, an automatic exposure control unit 602 that calculates the tube current profile of an X-ray radiation dose of the examined site according to the three-dimensional scan image and attenuation information, and a second scan unit 603 that performs a main scan of the examined site according to the tube current profile to obtain a main scan image.

In some embodiments, the X-ray radiation dose used for the three-dimensional scan is within 4% of the X-ray radiation dose used for the main scan. In some embodiments, the difference between the X-ray radiation dose used for the three-dimensional scan and 1% of the X-ray radiation dose used for the main scan is within a predetermined range. In some embodiments, the pitch factor of a pitch used for the three-dimensional scan is greater than 1.0.

In some embodiments, as shown in FIG. 6, the apparatus 600 further includes an extraction unit 604 that extracts the attenuation information for all angles in the scan range from raw scan data and the three-dimensional scout scan image obtained by carrying out the three-dimensional scan of the examined site.

In some embodiments, as shown in FIG. 6, the apparatus 600 further includes a segmentation unit 605 that performs organ segmentation on the three-dimensional scan image to generate a three-dimensional organ picture. The AEC unit 602 calculates the tube current profile along the scan range according to the inputted three-dimensional organ picture and the attenuation information corresponding to the three-dimensional organ picture. In some embodiments, the three-dimensional scan is a spiral scan.

For the sake of simplicity, FIG. 6 only exemplarily illustrates the connective relationship or signal direction between various components or modules, but it should be clear to those skilled in the art that various related technologies such as a bus connection can be used. The various components or modules can be implemented by means of a hardware facility such as a processor and a memory, etc. The embodiments of the present application are not limited thereto.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to the embodiments of the present application, the problem of patient size ambiguity in an image caused by improper centering during a two-dimensional scout scan is prevented by replacing two-dimensional scout scans with three dimensional scans, thereby providing precise three-dimensional organ segmentation for organ-based mA (X-ray tube current) modulation and organ protection, and avoiding the problem of difficulty in organ segmentation in the image. Moreover, a three-dimensional scanning mechanism using an ultra-low dose and a high pitch eliminates the problem of patient size ambiguity, providing additional volume information for mA profile estimation at a smaller dose increase cost.

An embodiment of the present application provides an electronic device, including the computed tomography imaging apparatus 600 as described in the embodiment of the second aspect, the contents of which are incorporated herein. The electronic apparatus may, for example, be a computer, a server, a workstation, a laptop, a smart phone, etc., but the embodiments of the present application are not limited thereto.

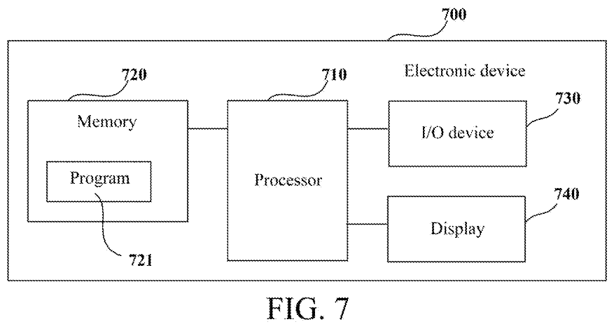

FIG. 7 is a schematic diagram of an electronic apparatus according to an embodiment of the present application. As shown in FIG. 7, the electronic apparatus 700 may include one or more processors (for example, a central processing unit (CPU)) 710, and one or more memories 720, the memory 720 being coupled to the one or more processors 710. Here, the memory 720 can store various pieces of data, further stores a program 721 for information processing, and executes the program 721 under the control of the processor 710.

In some embodiments, functions of the computed tomography imaging apparatus 600 are integrated into and implemented by the processor 710. Here, the processor 710 is configured to implement the computed tomography imaging method according to the embodiments of the first aspect.

In some embodiments, the computed tomography imaging apparatus 600 and the processor 710 are configured separately. For example, the computed tomography imaging apparatus 600 can be configured to be a chip that is connected to the processor 710 and the functions of the computed tomography imaging apparatus 600 can be achieved by means of the control of the processor 710.

For example, the processor 710 is configured to perform the following controls: performing a three-dimensional scan of an examined site to obtain a three-dimensional scan image of the examined site and attenuation information of the examined site, the three-dimensional scan image being used for positioning a scan range; calculating the tube current profile of an X-ray radiation dose of the examined site according to the three-dimensional scan image and attenuation information; and performing a main scan of the examined site according to the tube current profile to obtain a main scan image.

In addition, as shown in FIG. 7, the electronic device 700 may further include: an input/output (I/O) device 730 and a display 740, etc. Functions of the above components are similar to those in the prior art, and are not repeated herein. It should be noted that the electronic device 700 does not necessarily include all of the components shown in FIG. 7. In addition, the electronic device 700 may further include components not shown in FIG. 7, for which reference may be made to related technology.

Embodiments of the present application further provide a computer-readable program, wherein when a program is executed in an electronic apparatus, the program enables a computer to execute, in the electronic apparatus, the computed tomography imaging method as described in the embodiments of the first aspect.

Embodiments of the present application further provide a storage medium that stores a computer-readable program, wherein the computer-readable program enables a computer to execute, in the electronic device, the computed tomography imaging method as described in the embodiments of the first aspect.

The above apparatus and method of the present application can be implemented by hardware, or can be implemented by hardware in combination with software. The present application relates to the foregoing type of computer-readable program. When executed by a logic component, the program causes the logic component to implement the foregoing device or a constituent component, or causes the logic component to implement various methods or steps as described above. The present application further relates to a storage medium for storing the above program, such as a hard disk, a disk, an optical disk, a DVD, a flash memory, etc.

The method/device described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A computed tomography imaging method, comprising:
performing a three-dimensional scan of an examined site to obtain a three-dimensional scan image of the examined site and attenuation information of the examined site, wherein the three-dimensional scan image is used to position a scan range;
calculating a tube current profile of an X-ray radiation dose of the examined site according to the three-dimensional scan image and attenuation information; and
performing a main scan of the examined site according to the tube current profile to obtain a main scan image.

2. The method according to claim 1, wherein, the X-ray radiation dose used for the three-dimensional scan is within 4% of the X-ray radiation dose used for the main scan.

3. The method according to claim 1, wherein, the difference between the X-ray radiation dose used for the three-dimensional scan and 1% of the X-ray radiation dose used for the main scan is within a predetermined range.

4. The method according to claim 1, wherein, the pitch factor of a pitch used for the three-dimensional scan is greater than 1.0.

5. The method according to claim 1, further comprising:
extracting attenuation information for all angles in the scan range from raw scan data and the three-dimensional scan image obtained by carrying out the three-dimensional scan of the examined site.

6. The method according to claim 1, wherein calculating the tube current profile further comprises:
performing organ segmentation on the three-dimensional scan image to generate a three-dimensional organ picture; and
calculating the tube current profile along the scan range according to the three-dimensional organ picture and attenuation information corresponding to the three-dimensional organ picture.

7. The method according to any one of claim 1, wherein, the three-dimensional scan is a spiral scan.

8. A computed tomography imaging apparatus, comprising:
a first scan unit that performs a three-dimensional scan of an examined site to obtain a three-dimensional scan image of the examined site and attenuation information of the examined site, the three-dimensional scan image being used to position a scan range;
an automatic exposure control unit that calculates a tube current profile of an X-ray radiation dose of the examined site according to a three-dimensional scout scan image and the attenuation information; and
a second scan unit that performs a main scan of the examined site according to the tube current profile to obtain a main scan image.

9. The apparatus according to claim 8, wherein the X-ray radiation dose used for the three-dimensional scan is within 4% of the X-ray radiation dose used for the main scan.

10. The apparatus according to claim 8, wherein the difference between the X-ray radiation dose used for the three-dimensional scan and 1% of the X-ray radiation dose used for the main scan is within a predetermined range.

11. The apparatus according to claim 8, wherein the pitch factor of a pitch used for the three-dimensional scan is greater than 1.0.

12. The apparatus according to claim 8, further comprising:
an extraction unit that extracts the attenuation information for all angles in the scan range from raw scan data and the three-dimensional scout scan image obtained by carrying out the three-dimensional scan of the examined site.

13. The apparatus according to claim 8, further comprising:
a segmentation unit that performs organ segmentation on the three-dimensional scan image to generate a three-dimensional organ picture; and
the automatic exposure control unit that calculates the tube current profile along the scan range according to the three-dimensional organ picture and attenuation information corresponding to the three-dimensional organ picture.

14. The apparatus according to any one of claim 8, wherein the three-dimensional scan is a spiral scan.
